SEQUENCE LISTING (10 sequences)

SEQ ID NO: 1 - DNA, 993 bp, Escherichia coli
  CDS (1)..(990): rpoS gene
atgagtcagaatacgctgaaagttcatgatttaaatgaagatgcggaatttgatgagaacggagttgaggtttttgacgaaaaggccttagtagaacaggaacccagtgataacgatttggccgaagaggaactgttatcgcagggagccacacagcgtgtgttggacgcgactcagctttaccttggtgagattggttattcaccactgttaacggccgaagaagaagtttattttgcgcgtcgcgcactgcgtggagatgtcgcctctcgccgccggatgatcgagagtaacttgcgtctggtggtaaaaattgcccgccgttatggcaatcgtggtctggcgttgctggaccttatcgaagagggcaacctggggctgatccgcgcggtagagaagtttgacccggaacgtggtttccgcttctcaacatacgcaacctggtggattcgccagacgattgaacgggcgattatgaaccaaacccgtactattcgtttgccgattcacatcgtaaaggagctgaacgtttacctgcgaaccgcacgtgagttgtcccataagctggaccatgaaccaagtgcggaagagatcgcagagcaactggataagccagttgatgacgtcagccgtatgcttcgtcttaacgagcgcattacctcggtagacaccccgctgggtggtgattccgaaaaagcgttgctggacatcctggccgatgaaaaagagaacggtccggaagataccacgcaagatgacgatatgaagcagagcatcgtcaaatggctgttcgagctgaacgccaaacagcgtgaagtgctggcacgtcgattcggtttgctggggtacgaagcggcaacactggaagatgtaggtcgtgaaattggcctcacccgtgaacgtgttcgccagattcaggttgaaggcctgcgccgtttgcgcgaaatcctgcaaacgcaggggctgaatatcgaagcgctgttccgcgagtaa

SEQ ID NO: 2 - protein, 330 aa, Escherichia coli
MSQNTLKVHDLNEDAEFDENGVEVFDEKALVEQEPSDNDLAEEELLSQGATQRVLDATQLYLGEIGYSPLLTAEEEVYFARRALRGDVASRRRMIESNLRLVVKIARRYGNRGLALLDLIEEGNLGLIRAVEKFDPERGFRFSTYATWWIRQTIERAIMNQTRTIRLPIHIVKELNVYLRTARELSHKLDHEPSAEEIAEQLDKPVDDVSRMLRLNERITSVDTPLGGDSEKALLDILADEKENGPEDTTQDDDMKQSIVKWLFELNAKQREVLARRFGLLGYEAATLEDVGREIGLTRERVRQIQVEGLRRLREILQTQGLNIEALFRE

SEQ ID NO: 3 - DNA, 993 bp, Escherichia coli
  Allele (1)..(990): rpoS allele
atgagtcagaatacgctgaaagttcatgatttaaatgaagatgcggaatttgatgagaacggagttgaggtttttgacgaaaaggccttagtagaataggaacccagtgataacgatttggccgaagaggaactgttatcgcagggagccacacagcgtgtgttggacgcgactcagctttaccttggtgagattggttattcaccactgttaacggccgaagaagaagtttattttgcgcgtcgcgcactgcgtggagatgtcgcctctcgccgccggatgatcgagagtaacttgcgtctggtggtaaaaattgcccgccgttatggcaatcgtggtctggcgttgctggaccttatcgaagagggcaacctggggctgatccgcgcggtagagaagtttgacccggaacgtggtttccgcttctcaacatacgcaacctggtggattcgccagacgattgaacgggcgattatgaaccaaacccgtactattcgtttgccgattcacatcgtaaaggagctgaacgtttacctgcgaaccgcacgtgagttgtcccataagctggaccatgaaccaagtgcggaagagatcgcagagcaactggataagccagttgatgacgtcagccgtatgcttcgtcttaacgagcgcattacctcggtagacaccccgctgggtggtgattccgaaaaagcgttgctggacatcctggccgatgaaaaagagaacggtccggaagataccacgcaagatgacgatatgaagcagagcatcgtcaaatggctgttcgagctgaacgccaaacagcgtgaagtgctggcacgtcgattcggtttgctggggtacgaagcggcaacactggaagatgtaggtcgtgaaattggcctcacccgtgaacgtgttcgccagattcaggttgaaggcctgcgccgtttgcgcgaaatcctgcaaacgcaggggctgaatatcgaagcgctgttccgcgagtaa

SEQ ID NO: 4 - DNA, 75 bp, Escherichia coli
  tRNA (1)..(75): supE allele
tggggtatcgccaagcggtaaggcaccggattctaattccggcattccgaggttcgaatcctcgtaccccagcca

SEQ ID NO: 5 - DNA, 1545 bp, Escherichia coli
  CDS (1)..(1542): ilvA gene
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 6 - protein, 514 aa, Escherichia coli [sequence omitted]

SEQ ID NO: 7 - DNA, 1545 bp, Escherichia coli
  CDS (1)..(1542): ilvA allele
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcgaaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 8 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDAVRGFGGEVLLHGANFDEAKAKAIELSQQQGFTWVPPFDHPMVIAGQGTLALELLQQDAHLDRVFVPVGGGGLAAGVAVLIKQLMPQIKVIAVEAEDSACLKAALDAGHPVDLPRVGLFAEGVAVKRIGDETFRLCQEYLDDIITVDSDAICAAMKDLFEDVRAVAKPSGALALAGMKKYIALHNIRGERLAHILSGANVNFHGLRYVSERCELGEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFELGDHEPDFETRLNELGYDCHDETNNPAFRFFLAG

SEQ ID NO: 9 - DNA, 1548 bp, Escherichia coli
  DNA (1)..(1548)
tcgcgatctggtactgtaaggggaaatagagatgacacacgataataaattgcaggttgaagctattaaacgcggcacggtaattgaccatatccccgcccagatcggttttaagctgttgagtctgttcaagctgaccgaaacggatcagcgcatcaccattggtctgaacctgccttctggcgagatgggccgcaaagatctgatcaaaatcgaaaatacctttttgagtgaagatcaagtagatcaactggcattgtatgcgccgcaagccacggttaaccgtatcgacaactatgaagtggtgggtaaatcgcgcccaagtctgccggagcgcatcgacaatgtgctggtctgcccgaacagcaactgtatcagccatgccgaaccggtttcatccagctttgccgtgcgaaaacgcgccaatgatatcgcgctcaaatgcaaatactgtgaaaaagagttttcccataatgtggtgctggccaattaattgcggttggtaataaaagtctggctccctataatgagccagactttttaccgctgtaataaaggagaaatcatgagcaaaactatcgcgacggaaaatgcaccggcagctatcggtccttacgtacagggcgttgatctgggcaatatgatcatcacctccggtcagatcccggtaaatccgaaaacgggcgaagtaccggcagacgtcgctgcacaggcacgtcagtcgctggataacgtaaaagcgatcgtcgaagccgctggcctgaaagtgggcgacatcgttaaaactaccgtgtttgtaaaagatctgaacgacttcgcaaccgtaaacgccacttacgaagccttcttcaccgaacacaacgccaccttcccggcacgttcttgcgttgaagttgcccgtctgccgaaagacgtgaagattgagatcgaagcgatcgctgttcgtcgctaatcttgatggaaatccgggctatcatgcccggattaagtctgatgacaaacgcaaaatcgcctgatgcgctacgcttatcaggcctacgtgattcctgcaatttattgaatttgttggccggataaggcatttacgccgcatccggcatgaacaaaactcactttgtctacaatctgaatcggggctatcgtgcccagtttattctttattgccagccgtaacgacggctatagaaccctttcaccaactgggttaatgtcatataccctgccagaatcgcaaccagccacgggaaatagcttaacggcagcgcctgtaattgcagataactggccagcggtgaaaacggcaatgcgatcccgacaatcatcacgatcacggtcatgatcattaacggccacgatgcacagctctgaataaacggcacacggcgggtgcggatcatatgcacaatcagcgtttgcgacagtaagcccaccacaaaccatcccgactggaacagcgtttgcgtttccggcgtgttggcatggaatacccaccacatcaggcaaaacgtcaaaatatcgaagatcgagctgatcggtccgaagaagatc

SEQ ID NO: 10 - protein, 141 aa, Escherichia coli
MSQTFYRCNKGEIMSKTIATENAPAAIGPYVQGVDLGNMIITSGQIPVNPKTGEVPADVAAQARQSLDNVKAIVEAAGLKVGDIVKTTVFVKDLNDFATVNATYEAFFTEHNATFPARSCVEVARLPKDVKIEIEAIAVRR

(19) United States
(12) Patent Application Publication
(10) Pub. No.: US 2006/0252133 A1
(43) Pub. Date: Nov. 9, 2006

(54) Process for the preparation of l-threonine

(75) Inventors: Daniela Kruse, Bielefeld (DE); Thomas Hermann, Bielefeld (DE); Georg Thierbach, Bielefeld (DE); Mechthild Rieping, Bielefeld (DE)

Correspondence Address: FITCH, EVEN, TABIN & FLANNERY, P. O. BOX 65973, WASHINGTON, DC, 20035, US

(73) Assignee: Degussa AG, Duseldorf (DE)

(21) Appl. No.: 10/566,606

(22) PCT Filed: Jul. 27, 2004
(86) PCT No.: PCT/EP04/08390

§ 371 Date: May 11, 2006

(30) Foreign Application Priority Data
Aug. 1, 2003 (DE) 103352538
Aug. 4, 2003 (US) 60491981
May 11, 2004 (DE) 1020040230552
Jun. 15, 2004 (DE) 1020040288593

(51) Int. Cl. C12P 13/04; C12N 1/21

(52) U.S. Cl. 435/106; 435/252.33

(57) ABSTRACT

The invention relates to an improved process for the fermentative preparation of L-threonine using bacteria of the Enterobacteriaceae family which produce L-threonine.

### CROSS REFERENCE TO RELATES APPLICATIONS

The present application represents U.S. national stage of international application PCT/EP2004/008390, which has an international filing date of Jul. 27, 2004, and which was published in English under PCT Article 21(2) on Feb. 17, 2005. The international application claims priority to German applications 103 35 253.8, filed on Aug. 1, 2003; 10 2004 023 055.2, filed on May 11, 2004; and 10 2004 028 859.3, filed on Jun. 15, 2004. In addition, the international application claims priority to U.S. provisional application 60/491,981, filed on Aug. 4, 2003. These prior applications are hereby incorporated by reference in their entirety.

### FIELD OF THE INVENTION

The invention relates to an improved process for the fermentative preparation of L-threonine using bacteria of the Enterobacteriaceae family.

### BACKGROUND OF THE INVENTION

L-Threonine is used in animal nutrition, in human medicine and in the pharmaceuticals industry.

It is known that L-threonine can be prepared by fermentation of strains of the Enterobacteriaceae family, in particular Escherichia coli. Because of the great importance of this amino acid, work is constantly being undertaken to improve the preparation processes. Improvements to the process can relate to fermentation measures, such as e.g. stirring and supply of oxygen, or the composition of the nutrient media, such as e.g. the sugar concentration during the fermentation, or the working up to the product form, by e.g. ion exchange-chromatography, or the intrinsic output properties, i.e. those of genetic origin, of the microorganism itself.

It is known that threonine can be prepared by fermentation of bacteria of the Enterobacteriaceae family, in particular Escherichia coli, in the batch process or fed batch process. In the batch process, all the nutrients are initially introduced directly at the start of the fermentation. In the fed batch process an additional nutrient medium is fed to the culture. This feed can start directly at the start of culturing or after a certain culturing time has elapsed, for example when a component introduced with the first nutrient medium initially introduced has been consumed. At the end of the fermentation, the complete contents of the ferments are harvested and the threonine contained in the fermentation broth is isolated and purified or otherwise processed. This process is described, for example, in the patent specifications US 5,538,873, EP-B-0593792 and WO 01/4525 and by Okamoto et al. (Bioscience, Biotechnology, and Biochemistry 61 (11), 1877-1882, 1997).

Another process for the preparation of threonine using bacteria of the Enterobacteriaceae family, in particular Escherichia coli, is described in the patent specification U.S. Pat. No. 6,562,601. It comprises initially culturing the bacterium by the fed batch process, threonine becoming concentrated in the fermentation broth. At a desired point in time, a portion, i.e. 10 to 99%, of the fermentation broth contained in the fermenter is harvested. The remaining portion of the fermentation broth remains in the fermenter. The fermentation broth remaining in the fermenting tank is topped up with nutrient medium and a further fermentation is carried out by the fed batch process. The cycle described is optionally carried out several times.

### OBJECT OF THE INVENTION

It was the object of this invention to provide new measures for improved fermentative preparation of L-threonine.

### SUMMARY OF THE INVENTION

The invention provides a fermentation process, which is wherein
a) a bacterium of the Enterobacteriaceae family which produces L-threonine is inoculated and cultured in at least a first nutrient medium,
b) at least a further nutrient medium or several further nutrient media is/are then fed continuously to the culture in one or several feed streams, the further nutrient medium or the further nutrient media comprising at least one source of carbon, at least one source of nitrogen and at least one source of phosphorus, under conditions which allow the formation of L-threonine, and at the same time culture broth is removed from the culture with at least one or several removal streams which substantially corresponds/correspond to the feed stream or the total of the feed streams, wherein
c) the concentration of the source(s) of carbon during the continuous culturing is adjusted to not more than 30 g/l

### DETAILED DESCRIPTION OF THE INVENTION

According to the invention, the plant output of a fermenter which produces L-threonine can be increased by culturing by the batch process or fed batch process in the first step a) described above, at least one additional nutrient medium being employed if the fed batch process is used. In the subsequent step b) described, at least one further nutrient medium or several further nutrient media are fed continuously to the culture in one or several feed streams and at the same time culture broth is removed from the culture with at least one or several removal streams, which substantially corresponds/correspond to the feed stream or the total of the feed streams.

The term plant output is understood as meaning that in a plant, such as e.g. a fermenter, the weight or amount of a product, e.g. L-threonine, is prepared with a certain yield and with a certain rate or productivity or space/time yield. These parameters largely determine the costs or the profitability of a process.

A culture broth is understood as meaning the suspension of a microorganism formed by culturing a microorganism—in the case of the present invention a bacterium which produces L-threonine—in a nutrient medium using a fermenter or culture vessel.

During the step a), the bacterium is inoculated in at least a first nutrient medium and cultured by the-batch process or fed batch process. If the fed batch process is used, an additional nutrient medium is fed in after more than 0 to not more than 10 hours, preferably after 1 to 10 hours, preferentially after 2 to 10 hours and particularly preferably after 3 to 7 hours.

The first nutrient medium comprises as the source of carbon one or more of the compounds chosen from the group consisting of sucrose, molasses from sugar beet or cane sugar, fructose, glucose, starch hydrolysate, lactose, galactose, maltose, xylose, cellulose hydrolysate, arabinose, acetic acid, ethanol and methanol, in concentrations of 1 to 100 g/kg or 1 to 50 g/kg, preferably 10 to 45 g/kg, particularly preferably 20 to 40 g/kg. Starch hydrolysate is understood according to the invention as the hydrolysis product of starch from maize, cereals, potatoes or tapioca.

Organic nitrogen-containing compounds, such as peptones, yeast extract, meat extract, malt extract, corn steep liquor, soya bean flour and urea, or inorganic compounds, such as ammonia, ammonium sulfate, ammonium chloride, ammonium phosphate, ammonium carbonate and ammonium nitrate, potassium nitrate and potassium sodium nitrate, can be used as the source of nitrogen in the first nutrient medium. The sources of nitrogen can be used individually or as a mixture in concentrations of 1 to 40 g/kg, preferably 10 to 30 g/kg, particularly preferably 10 to 25 g/kg, very particularly preferably 1 to 30 g/kg or 1 to 25 g/kg.

Phosphoric acid, alkali metal or alkaline earth metal salts of phosphoric acid, in particular potassium dihydrogen phosphate or dipotassium hydrogen phosphate or the corresponding sodium-containing salts, polymers of phosphoric acid or the hexaphosphoric acid ester of inositol, also called phytic acid, or alkali metal or alkaline earth metal salts thereof, can be used as the source of phosphorus in the first nutrient medium in concentrations of 0.1 to 5 g/kg, preferably 0.3 to 3 g/kg, particularly preferably 0.5 to 1.5 g/kg. The first nutrient medium must furthermore comprise salts of metals, such as e.g. magnesium sulfate or iron sulfate, which are necessary for growth. These substances are present in concentrations of 0.003 to 3 g/kg. Finally, essential growth substances, such as amino acids (e.g. homoserine) and vitamins (e.g. thiamine), are employed in addition to the above-mentioned substances. Antifoams, such as e.g. fatty acid polyglycol esters, can be employed to control the development of foam.

The additional nutrient medium which is used in a fed batch process in general comprises merely as the source of carbon one or more of the compounds chosen from the group consisting of sucrose, molasses from sugar beet or cane sugar, fructose, glucose, starch hydrolysate, lactose, galactose, maltose, xylose, cellulose hydrolysate, arabinose, acetic acid, ethanol and methanol, in concentrations of 300 to 700 g/kg, preferably 400 to 650 g/kg, and optionally an inorganic source of nitrogen, such as e.g. ammonia, ammonium sulfate, ammonium chloride, ammonium phosphate, ammonium carbonate, ammonium nitrate, potassium nitrate or potassium sodium nitrate. Alternatively, these and other components can also be fed in separately.

It has been found that in the continuous culturing according to step b) the constituents of the further nutrient medium can be fed to the culture in the form of a single further nutrient medium and in a plurality of further nutrient media. According to the invention, the further nutrient medium or the further nutrient media is or are fed to the culture in at least one (1) feed stream or in a plurality of feed streams of at least 2 to 10, preferably 2 to 7 or 2 to 5 feed streams.

The term “continuous” means that the feed stream or the feed streams is/are added substantially without interruption, that is to say with at most short, individual pauses, to the culture. The individual interruptions or pauses are up to a maximum of 0.1, 1, 2 or 3 hours. The sum of the individual interruptions or pauses in the continuous culturing according to step b) is a maximum of 10%, 8%, 6%, 4%, 2% or 1% of the total time of the continuous culturing according to step b).

The further nutrient medium or the further nutrient media comprises/comprise as the source of carbon one or more of the compounds chosen from the group consisting of sucrose, molasses from sugar beet or cane sugar, fructose, glucose, starch hydrolysate, maltose, xylose, cellulose hydrolysate, arabinose, acetic acid, ethanol and methanol, in concentrations of 20 to 700 g/kg, preferably 50 to 650 g/kg.

The further nutrient medium or the further nutrient media furthermore comprises or comprise a source of nitrogen consisting of organic nitrogen-containing compounds, such as peptones, yeast extract, meat extract, malt extract, corn steep liquor, soya bean flour and urea, or inorganic compounds, such as ammonia, ammonium sulfate, ammonium chloride, ammonium phosphate, ammonium carbonate, ammonium nitrate and/or potassium nitrate or potassium sodium nitrate. The sources of nitrogen can be used individually or as a mixture in concentrations of 5 to 50 g/kg, preferably 10 to 40 g/kg.

The further nutrient medium or the further nutrient media furthermore comprises or comprise a source of phosphorus consisting of phosphoric acid or the alkali metal or alkaline earth metal salts of phosphoric acid, in particular potassium dihydrogen phosphate or dipotassium hydrogen phosphate or the corresponding sodium-containing salts, polymers of phosphoric acid or the hexaphosphoric acid ester of inositol, also called phytic acid, or the corresponding alkali metal or alkaline earth metal salts. The sources of phosphorus can be used individually or as a mixture in concentrations of 0.3 to 3 g/kg, preferably 0.5 to 2 g/kg. The further nutrient medium or the further nutrient media must furthermore comprise salts of metals, such as e.g. magnesium sulfate or iron sulfate, which are necessary for growth, in concentrations of 0.003 to 3 g/kg, preferably in concentrations of 0.008 to 2 g/kg. Finally, essential growth substances, such as amino acids (e.g. homoserine) and vitamins (e.g. thiamine), are employed in addition to the above-mentioned substances. Antifoams, such as e.g. fatty acid polyglycol esters, can be employed to control the development of foam.

If a single further nutrient medium is used, this is typically fed to the culture in one feed stream. If a plurality of further nutrient media are used, these are fed in a corresponding plurality of feed streams. If a plurality of further nutrient media are used, it should be noted that these in each case can comprise only one of the sources of carbon, nitrogen or phosphorus described, or also a mixture of the sources of carbon, nitrogen or phosphorus described.

According to the invention, the further nutrient medium fed in or the further nutrient media fed in is/are adjusted such that there is a phosphorus to carbon ratio (P/C ratio) of not more than 4; of not more than 3; of not more than 2; of not more than 1.5; of not more than 1; of not more than 0.7; of not more than 0.5; of not more than 0.48; of not more than 0.46; of not more than 0.44; of not more than 0.42; of not more than 0.40; of not more than 0.38; of not more than 0.36; of not more than 0.34; of not more than 0.32; or of not more than 0.30 mmol of phosphorus/mol of carbon.

The feed stream or the total of the feed streams in the process according to the invention are fed in at a rate corresponding to an average residence time of less than 30 hours, preferably less than 25, very particularly preferably less than 20 hours. The average residence time here is the theoretical time the particles remain in a continuously operated culture. The average residence time is described by the ratio of the volume of liquid in the reactor and the amount flowing through (Biotechnologie [Biotechnology], H. Weide, J. Páca and W. A. Knorre, Gustav Fischer Verlag Jena, 1991).

Intensive growth at the start of culturing according to step (a) is usually a logarithmic growth phase. The logarithmic growth phase is in general followed by a phase of less intensive cell growth than in the logarithmic phase.

If the process according to the invention starts in step a) with a batch process, after >(more than) 0 to 20 hours, 1 to 20 hours, after 1 to 10 hours, 2 to 10 hours or 3 to 7 hours, with respect to the start of the batch process, a further nutrient medium or further nutrient media is/are fed to the culture in one or more feed streams. The start of the removal of the culture broth with one or more removal streams takes place with the start of the feeding in of the further nutrient medium or the further nutrient media or with a time shift, i.e. before or after the start of feeding in of the further nutrient medium or the further nutrient media. If the start of the feeding in and the start of the removal take place with a time shift, the corresponding time difference is in general a maximum of 5 hours, 3 hours, 2 hours or 1 hour.

If the process according to the invention starts in step a) with a fed batch process, after > (more than) 0 to 80 hours, 1 to 80 hours, after 1 to 60 hours, 5 to 50 hours, 6 to 45 hours, or 8 to 40 hours, with respect to the start of the fed batch process, a further nutrient medium or further nutrient media is/are added to the culture in one or more feed streams. The start of the removal of the culture broth with one or more removal streams takes place with the start of the feeding in of the further nutrient medium or the further nutrient media or with a time shift, i.e. before or after the start of feeding in of the further nutrient medium. If the start of the feeding in and the start of the removal take place with a time shift, the corresponding time difference is in general a maximum of 5 hours, a maximum of 3 hours, a maximum of 2 hours or a maximum of 1 hour.

After > (more than) 0 to 100 hours, 1 to 85 hours, 2 to 80 hours, 3 to 75 hours, 5 to 72 hours, 10 to 72 hours, 10 to 60 hours, or 15 to 48 hours, with respect to the start of the process according to the invention according to step (a), the removal stream or the total of the removal streams substantially corresponds to the feed stream or the total of the feed streams and the state of continuous culturing according to step b) of the process according to the invention is reached. Substantially here means that the speed of the removal stream or the removal streams corresponds to 80%-120%, 90%-110% or 95%-105% of the feed stream or of the total of the feed streams. The removal can be realized industrially by pumping off and/or by draining off the culture broth.

According to the invention, the concentration of the source of carbon at least during the continuous culturing according to step (b) is in general adjusted to not more than 30 g/l, not more than 20 g/l, not more than 10 g/l, preferably to not more than 5 g/l, particularly preferably not more than 2 g/l. This concentration is maintained at least during 75%, preferably at least during 85%, particularly preferably at least during 95% of time of culturing according to step (b). The concentration of the source of carbon is determined here with the aid of methods which are prior art. β-D-Glucose is determined e.g. in a YSI 02700 Select glucose analyzer from Yellow Springs Instruments (Yellow Springs, Ohio, USA).

If appropriate, the culture broth removed can be provided with oxygen or an oxygen-containing gas until the concentration of the source of carbon falls below 2 g/l, below 1 g/l or below 0.5 g/l.

In a process according to the invention, the yield is at least 31%, at least 33%, at least 35%, at least 37%, at least 38%, at least 40%, at least 42%, at least 44%, at least 46% or at least 48%. The yield is defined here as the ratio of the total amount of L-threonine formed in a culturing to the total amount of the source of carbon employed or consumed.

In a process according to the invention, L-threonine is formed with a space/time yield of at least 1.5 to 2.5 g/l per h, of at least 2.5 to 3.5 g/l per h, of at least 2.5 to more than 3.5 g/l per h, of at least 3.5 to 5.0 g/l per h, of at least 3.5 to more than 5.0 g/l per h, or of at least 5.0 to 8.0 g/l or more per h. The space/time yield is defined here as the ratio of the total amount of threonine formed in a culturing to the volume of the culture over the total period of time of culturing. The space/time yield is also called the volumetric productivity.

In a fermentation process like that according to the invention, the product is of course prepared in a certain yield and in a certain space/time yield (volumetric productivity). In a process according to the invention, L-threonine can be prepared in a yield of at least 31 wt. % and a space/time yield of at least 1.5 to 2.5 g/l per h. Further couplings of yield with space/time yield, such as, for example, a yield of at least 37% and a space/time yield of at least 2.5 g/l per h, automatically result from the above statements.

During the culturing the temperature is adjusted in a range from 29 to 42° C., preferably 33 to 40° C. The culturing can be carried out under normal pressure or optionally under increased pressure, preferably under an increased pressure of 0 to 1.5 bar. The oxygen partial pressure is regulated at 5 to 50%, preferably approx. 20% atmospheric saturation. During this procedure the culture is stirred and supplied with oxygen. Regulation of the pH to a pH of approx. 6 to 8, preferably 6.5 to 7.5, can be effected with 25% aqueous ammonia.

The process according to the invention is operated for at least approx. 72 hours, preferably 100 to ≧300, particularly preferably 200 to ≧300 hours. In the process according to the invention, the volume of the culture is exchanged at least by half, at least once, at least 2 times, at least 3 times, at least 4 times, at least 6 times, at least 8 times, at least 10 times, at least 12 times.

From the culture broth removed, the L-threonine can be isolated, collected or concentrated and optionally purified.

It is also possible to prepare a product from the culture broth removed (=fermentation broth) by removing the bacterium biomass contained in the culture broth completely (100%) or almost completely, i.e. more than or greater than (>) 90%, >95%, >97%, >99% and leaving the other constituents of the fermentation broth largely, i.e. to the extent of 30%-100%, 40%-100%, 50%-100%, 60%-100%, 70%-100%, 80%-100%, or 90%-100%, preferably greater than or equal to (>) 50%, >60%, >70%, >80%, >90% or >95% or also completely (100%) in the product.

Separation methods such as, for example, centrifugation, filtration, decanting, flocculation or a combination thereof are employed for the removal or separating off of the biomass.

The broth obtained is then thickened or concentrated by known methods, such as, for example, with the aid of a rotary evaporator, thin film evaporator, falling film evaporator, by reverse osmosis, by nanofiltration or a combination thereof.

This concentrated broth is then be worked up by methods of freeze drying, spray drying, spray granulation or by other processes to give a preferably free-flowing, finely divided powder. This free-flowing, finely divided powder can then in turn by converted by suitable compacting or granulating processes into a coarse-grained, readily free-flowing, storable and largely dust-free product. The water is removed in total to the extent of more than 90% by this means, so that the water content in the product is less than 10%, less than 5%.

The process steps mentioned do not necessarily have to be carried out in the sequence stated here, but can optionally be combined in an industrially appropriate manner.

The analysis of L-threonine and other amino acids can be carried out by anion exchange chromatography with subsequent ninhydrin derivation, as described by Spackman et al. (Analytical Chemistry, 30: 1190-1206 (1958)) or it can be carried out by reversed phase HPLC, as described by Lindroth et al. (Analytical Chemistry 51: 1167-1174 (1979)).

Bacteria of the Enterobacteriaceae family which produce L-threonine chosen from the genera Escherichia, Erwinia, Providencia and Serratia are suitable for carrying out the process according to the invention. The genera Escherichia and Serratia are preferred. Of the genus Escherichia in particular the species Escherichia coli and of the genus Serratia in particular the species Serratia marcescens are to be mentioned.

The bacteria contain at least one copy of a thrA gene or allele which codes for a threonine-insensitive aspartate kinase I—homoserine dehydrogenase I. In this connection, “feed back” resistant or also desensitized variants are referred to in the literature. Such bacteria are typically resistant to the threonine analogue α-amino-β-hydroxyvaleric acid (AHV)(Shiio and Nakamori, Agricultural and Biological Chemistry 33 (8), 1152-1160 (1969)). Biochemical studies on “feed back” resistant aspartate kinase I—homoserine dehydrogenase I variants are described, for example, by Cohen et al. (Biochemical and Biophysical Research Communications 19(4), 546-550 (1965)) and by Omori et al. (Journal of Bacteriology 175(3), 785-794 (1993)). If appropriate, the threonine-insensitive aspartate kinase I—homoserine dehydrogenase I is overexpressed.

Methods of overexpression are adequately described in the prior art—for example by Makrides et al. (Microbiological Reviews 60 (3), 512-538 (1996)). By using vectors, the number of copies is increased by at least one (1) copy. Vectors which can be used are plasmids such as are described, for example, in U.S. Pat. No. 5,538,873. Vectors which can also be used are phages, for example the phage Mu, as described in EP 0 332 448, or the phage lambda (λ). An increase in the number of copies can also be achieved by incorporating a further copy into a further site of the chromosome—for example into the att site of the phage λ (Yu and Court, Gene 223, 77-81 (1998)). U.S. Pat. No. 5,939,307 discloses that by incorporation of expression cassettes or promoters, such as, for example, the tac promoter, trp promoter, lpp promoter or P_{L} promoter and P_{R}-promoter of the phage λ, upstream of the chromosomal threonine operon it was possible to achieve an increase in the expression. The promoters of the phage T7, the gear-box promoters or the nar promoter can be used in the same way.

Such expression cassettes or promoters can also be used, as described in EP 0 593 792, to overexpress plasmid-bound genes. By using the lacI^{Q} allele, the expression of plasmid-bound genes can in turn be controlled (Glascock and Weickert, Gene 223, 221-231 (1998)). By removal of the attenuator of the threonine operon (Park et al., Biotechnology Letters 24, 1815-1819 (2002)) or by using the thr79-20 mutation (Gardner, Proceedings of the National Academy of Sciences, USA 76(4), 1706-1710 (1979)) or by mutation of the thrS gene, which codes for threonyl-t-RNA synthetase, as described by Johnson et al. (Journal of Bacteriology 129(1), 66-70 (1977) an overexpression can likewise be achieved. By the measures described, the intracellular concentration of the particular aspartate kinase I—homoserine dehydrogenase I protein variant is increased by at least 10% compared with the starting strain.

A suitable thrA-allele is described in U.S. Pat. No. 4,278,765 and is obtainable in the form of the strain MG442 from the Russian National Collection of Industrial Microorganisms (VKPM, Moscow, Russia) under the Accession Number CMIM B-1628. Other suitable thrA alleles are described in WO 00/09660 and WO 00/09661 and are obtainable from the Korean Culture Center of Microorganisms (KCCM, Seoul, Korea) under the Accession Numbers KCCM 10132 and KCCM 10133. A further suitable thrA allele is present in the strain H-4581, which is described in U.S. Pat. No. 4,996,147 and is obtainable under the Accession Number Ferm BP-1411 from the National Institute of Advanced Industrial Science and Technology (1-1-1 Higashi, Tsukuba Ibaraki, Japan). Finally, further thrA alleles are described in U.S. Pat. No. 3,580,810 and are obtainable in the form of the strains ATCC 21277 and ATCC 21278 deposited at the ATCC. A further allele is described in U.S. Pat. No. 3,622,453 and is obtainable from the ATCC in the form of the strain KY8284 under the Accession Number ATCC 21272. Furthermore, WO 02/064808 describes a further thrA allele which is deposited at the KCCM in the form of the strain pGmTN-PPC12 under the Accession Number KCCM 10236.

If appropriate, thrA alleles which code for “feed back” resistant aspartate kinase I—homoserine dehydrogenase I variants can be isolated using the adequately known methods of conventional mutagenesis of cells using mutagenic substances, for example N-methyl-N′-nitro-N-nitrosoguanidine (MNNG) or ethyl methanesulfonate (EMS) or mutagenic rays, for example UV rays, and subsequent selection of threonine analogues (for example AHV-resistant variants). Such mutagenesis methods are described, for example, by Shiio and Nakamori (Agricultural and Biological Chemistry 33 (8), 1152-1160 (1969)) or by Saint-Girons and Margerita (Molecular and General Genetics 162, 101-107 (1978)) or in the known handbook of J. H. Miller (A Short Course In Bacterial Genetics. A Laboratory Manual and Handbook for Escherichia coli and Related Bacteria, Cold Spring Harbor Laboratory Press, New York, USA, 1992), in particular on pages 135 to 156. Shiio and Nakamori, for example, treat a cell suspension of Escherichia coli with 0.5 mg/ml MNNG in a 0.1 M sodium phosphate buffer of pH 7 at room temperature (i.e. in general approx. 16 to 26° C.) for approx. 15 minutes to generate mutations. Miller recommends, for example, a treatment for 5 to 60 minutes with 30 μl EMS per 2 ml of cell suspension in 0.1 M TRIS buffer at pH 7.5 at a temperature of 37° C. These mutagenesis conditions can be modified in an obvious manner. Selection of AHV-resistant mutants takes place on minimal agar, which typically contains 2 to 10 mM AHV. The corresponding alleles can then be cloned and subjected to a sequence determination and the protein variants coded by these alleles can be subjected to a determination of the activity. If appropriate, the mutants produced can also be used directly. The word “directly” means that the mutants produced can be employed for the preparation of L-threonine in a process according to the invention or that further modifications can be carried out on these mutants to increase the output properties, such as, for example, attenuation of the threonine degradation or overexpression of the threonine operon.

Methods of in vitro mutagenesis such as are described, for example, in the known handbook by Sambrook et al. (Molecular Cloning, A Laboratory Manual, 2nd ed., Cold Spring Harbor Laboratory Press, Cold Spring Harbor, N.Y., USA, 1989) can also be used in the same way. Corresponding methods are also available commercially in the form of so-called “kits”, such as, for example, the “QuikChange Site-Directed Mutagenesis Kit” from Stratagene (La Jolla, USA) described by Papworth et al. (Strategies 9(3), 3-4 (1996)).

These mutagenesis methods can of course also be used on other genes, alleles or strains or objectives and tasks, such as, for example, the production and isolation of mutants which are resistant to L-threonine.

Those thrA alleles which code for aspartate kinase I—homoserine dehydrogenase I variants which, in the presence of 10 mM L-threonine, have at least 40%, at least 45%, at least 50%, at least 55% or at least 60% of the homoserine dehydrogenase activity and/or which, in the presence of 1 mM L-threonine, have at least 60%, at least 70%, at least 75% or at least 80% of the homoserine dehydrogenase activity, compared with the activity in the absence of L-threonine, are preferred. Where appropriate, the aspartate kinase activity of the aspartate kinase I—homoserine dehydrogenase I variants mentioned in the presence of 10 mM L-threonine is at least 60%, at least 65%, at least 70%, at least 75% or at least 80% of the activity in the absence of L-threonine.

Bacteria of the Enterobacteriaceae family which contain a stop codon chosen from the group consisting of opal, ochre and amber, preferably amber in the rpoS gene, and a t-RNA suppressor chosen from the group consisting of opal suppressor, ochre suppressor and amber suppressor, preferably amber suppressor, are moreover suitable. The amber mutation preferably lies at position 33 according to the amino acid sequence of the RpoS gene product. supE is preferably employed as the amber suppressor. These bacteria are described in PCT/EP02/02055. A strain which contains the mutation described in the rpoS gene and the suppressor supE is obtainable under the Accession Number DSM 15189 from the Deutsche Sammlung für Mikroorganismen und Zellkulturen [German Collection of Microorganisms and Cell Cultures (Braunschweig, Germany).

The nucleotide sequence of the rpoS gene can be found in the prior art. The nucleotide sequence of the rpoS gene corresponding to Accession No. AE000358 is shown as SEQ ID NO. 1. The amino acid sequence of the associated RpoS gene product or protein is shown in SEQ ID NO. 2. The nucleotide sequence of an rpoS allele which contains a stop codon of the amber type at the position of the nucleotide sequence corresponding to position 33 of the amino acid sequence of the RpoS gene product or protein, corresponding to SEQ ID NO. 1 or SEQ ID NO. 2 respectively, is reproduced in SEQ ID NO. 3. The suppressor supE is described in the prior art and is shown as SEQ ID NO. 4.

Bacteria of the Enterobacteriaceae family which are not capable of breaking down threonine or utilizing it as a source of nitrogen under aerobic culture conditions are moreover suitable. Aerobic culture conditions are understood as meaning those under which the oxygen partial pressure in the fermentation culture is greater than (>) 0% during 90%, preferably 95%, very particularly preferably 99% of the duration of the fermentation. Such a strain is, for example, the strain KY10935 described by Okamoto (Bioscience, Biotechnology and Biochemistry 61(11), 1877-1882 (1997)). Strains which are not capable of breaking down threonine, with splitting off of nitrogen, in general have an attenuated threonine dehydrogenase (EC 1.1.1.103), which is coded by the tdh gene. The enzyme has been described by Aronson et al. (The Journal of Biological Chemistry 264(9), 5226-5232 (1989)). Attenuated tdh genes are described, for example, by Ravnikar and Somerville (Journal of Bacteriology, 1986, 168(1), 434-436), in U.S. Pat. No. 5,705,371, in WO 02/26993 and by Komatsubara (Bioprocess Technology 19, 467-484 (1994)).

A suitable tdh allele is described in U.S. Pat. No. 5,538,873 and is obtainable in the form of the strain B-3996 under the Accession Number. 1876 from the Russian National Collection of Industrial Microorganisms (VKPM, Moscow, Russia). A further tdh allele is described in U.S. Pat. No. 5,939,307 and is obtainable in the form of the strain kat-13 under the Accession Number NRRL B-21593 from the Agriculture Research Service Patent Culture Collection (Peoria, Ill., USA). Finally, a tdh allele is described in WO 02/26993 and is deposited in the form of the strain TH21.97 under the Accession Number NRRL B-30318 at the NRRL. The allele tdh-1::cat1212, which codes for a defective threonine dehydrogenase, is obtainable from the E. coli Genetic Stock Center (New Haven, Conn., USA) under the Accession Number CGSC 6945.

Bacteria of the Enterobacteriaceae family which have an at least partial need for isoleucine (“leaky phenotype”) which can be compensated by addition of L-isoleucine in a concentration of at least 10, 20 or 50 mg/l or L-threonine in a concentration of at least 50, 100 or 500 mg/l are moreover suitable.

Need or auxotrophy is in general understood as meaning the fact that as a result of a mutation, a strain has completely lost a wild-type function, for example an enzyme activity, and requires the addition of a supplement, for example an amino acid, for growth. A partial need or partial auxotrophy is referred to if, as a result of a mutation, a wild-type function, for example the activity of an enzyme from the biosynthesis pathway of an amino acid, is impaired or attenuated but not eliminated completely. Strains with a partial need typically have, in the absence of the supplement, a growth rate which is reduced, i.e. greater than (>) 0% and less than (<) 90%, 50%, 25% or 10%, compared with the wild-type. In the literature, this relationship is also called “leaky” phenotype or “leakiness” (Griffiths et al.: An Introduction to Genetic Analysis. 6^{th} edition, 1996, Freeman and Company, New York, USA).

A strain with such a partial need for isoleucine is described, for example, in WO 01/14525 and deposited in the form of the strain DSM9906 under the Accession Number KCCM 10168 at the KCCM. Threonine-secreting or -producing strains with a need for isoleucine in general have an attenuated threonine deaminase (E.C. Number 4.3.1.19), which is coded by the ilvA gene. Threonine deaminase is also known by the name threonine dehydratase. An attenuated ilvA gene which effects a partial isoleucine auxotrophy is described, for example, in U.S. Pat. No. 4,278,765 and is obtainable in the form of the strain MG442, deposited under the Accession Number B-1682, at the VKPM.

A further attenuated ilvA gene is described, for example, in WO 00/09660 and deposited in the form of the strain DSM 9807 under the Accession Number KCCM-10132 at the KCCM. Further attenuated ilvA genes are described by Komatsubara (Bioprocess Technology 19, 467-484 (1994)).

The amino acid sequence of a suitable and new threonine deaminase comprises, for example, the sequence of SEQ ID NO. 6, which can contain any amino acid apart from glutamic acid at position 286. Exchange of glutamic acid for lysine is preferred (E286K).

The term “amino acid” means, in particular, the proteinogenic L-amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan, L-proline and L-arginine.

SEQ ID NO. 8 shows the amino acid sequence of a threonine deaminase which contains the amino acid lysine at position 286; the associated nucleotide sequence is shown as SEQ ID NO. 7. This contains the nucleobase adenine at position 856.

Another suitable threonine deaminase is the variant described by Lee et al. (Journal of Bacteriology 185 (18), 5442-5451 (2003)),in which serine is exchanged for phenylalanine at position 97 (S97F). Further suitable threonine deaminases are the variants described by Fischer and Eisenstein (Journal of Bacteriology 175 (20), 6605-6613 (1993)), which have at least one of the amino acid exchanges chosen from the group consisting of: exchange of asparagine at position 46 for aspartic acid (N46D), exchange of alanine at position 66 for valine (A66V), exchange of proline at position 156 for serine (P156S), exchange of glycine at position 248 for cysteine (G248C) and exchange of aspartic acid at position 266 for tyrosine (D266Y).

By insertion or deletion mutagenesis of at least one base pair or nucleotide or by insertion or deletion of at least one codon in the coding region or by incorporation of a stop codon by transition or transversion mutagenesis into the coding region of the ilvA gene, alleles in which the expression of the ilvA gene is in general completely eliminated can be isolated. This method can also be applied to other genes, alleles or open reading frames, such as, for example, the tdh gene, which codes for threonine dehydrogenase.

Bacteria of the Enterobacteriaceae family which are resistant in their growth towards inhibition by L-threonine and/or L-homoserine are moreover suitable. Threonine-resistant strains and the preparation thereof are described, for example, by Astaurova et al. (Prikladnaya Biokhimia Microbiologiya (1985),21(5), 485 as the English translation: Applied Biochemistry and Microbiology (1986), 21, 485-490)). The mutant described by Austaurova is resistant towards 40 mg/ml L-threonine. Furthermore, for example, the strain 472T23, which can grow in the presence of 5 mg/ml L-threonine and at the same time is resistant to L-homoserine, is described in U.S. Pat. No. 5,175,107. The strain 472T232 is obtainable under the Accession Number BKIIM B-2307 from the VKPM and under the number ATCC 9801 from the ATCC. Furthermore, the strain DSM 9807, which can grow on a solid nutrient medium which comprises 7% L-threonine, is described in WO 00/09660. The strain DSM 9807 is obtainable under the Accession Number KCCM-10132 from the KCCM. Finally, the strain DSM 9906, which can grow in a medium which comprises 60% to 70% of an L-threonine fermentation mother liquid, is described in WO 01/14525. The strain DSM 9906 is obtainable under the Accession Number KCCM-10168 from the KCCM.

It is known (see EP 0994 190 A2 and Livshits et al. (Research in Microbiology 154, 123-135 (2003)), that resistance to L-threonine and L-homoserine is brought about by enhancement of the rhtA gene. The enhancement can be achieved by increasing the number of copies of the gene or by use of the rhtA23 mutation.

EP 0 994 190 A2 discloses that the enhancement of the rhtB gene effects resistance to L-homoserine and L-threonine, in particular to L-homoserine, and improves threonine production. By overexpression of the RhtB gene product in a strain called N99, it was possible to increase the minimum inhibitory concentration from 250 μg/ml to 30,000 μg/ml. EP 1,013,765 A1 discloses that an enhancement of the rhtC gene causes resistance to L-threonine and improves threonine production. A strain which can grow on a minimal agar in the presence of a concentration of at least 30 mg/ml L-threonine is called resistant to L-threonine. It is furthermore disclosed that an enhancement of the rhtB gene effects resistance to L-homoserine and improves threonine production. A strain which can grow on a minimal agar in the presence of a concentration of at least 5 mg/ml L-homoserine is called resistant to L-homoserine. The patent application mentioned describes strains which are resistant to 10 mg/ml L-homoserine and resistant to 50 mg/ml L-threonine. U.S. Pat. No. 4,996,147 describes the strain H-4581, which is resistant to 15 g/l homoserine. The strain H-4581 is obtainable under the Accession Number FERM BP-1411 from the National Institute of Advanced Industrial Science and Technology.

EP 1 016 710 A2 discloses that an enhancement of the open reading frame or gene yfiK or yeaS effects resistance to L-threonine and L-homoserine. By overexpression of the YfiK gene product in a strain called TG1, it was possible to increase the minimum inhibitory concentration in respect of L-homoserine from 500 μg/ml to 1,000 μg/ml and in respect of L-threonine from 30,000 p/ml to 40,000 μg/ml. By overexpression of the YeaS gene product, it was possible to increase the minimum inhibitory concentration in respect of L-homoserine from 500 μg/ml to 1,000 μg/ml and in respect of L-threonine from 30,000 p/ml to 50,000 μg/ml. It is furthermore demonstrated in the patent application mentioned that threonine production is improved by overexpression of the YfiK gene product.

Strains which can grow in the presence of ≧ (at least) 5 g/l, ≧10, ≧20 g/l, ≧30 g/l, ≧40 g/l, ≧50 g/l, ≧60 g/l and ≧70 g/l L-threonine, i.e. are resistant to L-threonine, and are suitable for the preparation of L-threonine in a process according to the invention are prepared according to these technical instructions.

Strains which have at least the following features are suitable in particular for the process according to the invention:
a) a threonine-insensitive aspartate kinase I—homoserine dehydrogenase I, which is optionally present in overexpressed form, and
b) a stop codon chosen from the group consisting of opal, ochre and amber, preferably amber in the rpoS gene, and a t-RNA suppressor chosen from the group consisting of opal suppressor, ochre suppressor and amber suppressor, preferably amber suppressor.

Strains which have at least the following features are furthermore suitable in particular for the process according to the invention:
a) a threonine-insensitive aspartate kinase I—homoserine dehydrogenase I, which is optionally present in overexpressed form,
b) are not capable, under aerobic culture conditions, of breaking down threonine, preferably by attenuation of threonine dehydrogenase,
c) an at least partial need for isoleucine, and
d) growth in the presence of at least 5 g/l threonine.

Strains which have at least the following features are very particularly suitable for the process according to the invention:
a) a threonine-insensitive aspartate kinase I—homoserine dehydrogenase I, which is optionally present in overexpressed form,
b) a stop codon chosen from the group consisting of opal, ochre and amber, preferably amber in the rpoS gene, and a t-RNA suppressor chosen from the group consisting of opal suppressor, ochre suppressor and amber suppressor, preferably amber suppressor,
c) are not capable, under aerobic culture conditions, of breaking down threonine, preferably by attenuation of threonine dehydrogenase,
d) an at least partial need for isoleucine, and
e) growth in the presence of at least 5 g/l threonine.

Moreover, the bacteria employed for the process according to the invention can furthermore have one or more of the following features:
Attenuation of phosphoenol pyruvate carboxykinase (PEP carboxykinase), which is coded by the pckA gene, as described, for example, in WO 02/29080,
Attenuation of phosphoglucose isomerase, which is coded by the pgi gene (Froman et al. Molecular and General Genetics 217(l):126-31 (1989)).
Attenuation of the YtfP gene product, which is coded by the open reading frame ytfP, as described, for example, in WO 02/29080,
Attenuation of the YjfA gene product, which is coded by the open reading frame yjfA, as described, for example, in, WO 02/29080,
Attenuation of pyruvate oxidase, which is coded by the poxB gene, as described, for example, in WO 02/36797,
Attenuation of the YjgF gene product, which is coded by the open reading frame yjgF, as described, for example, in PCT/EP03/14271, The yjgF orf of Escherichia coli has been described by Wasinger V C. and Humphery-Smith I. (FEMS Microbiology Letters 169(2): 375-382 (1998)), Volz K. (Protein Science 8(11): 2428-2437 (1999)) and Parsons et al. (Biochemistry 42(1): 80-89 (2003)). The associated nucleotide or amino acid sequences are available under the Accession Number AE000495 in public databanks. For better clarity, these are shown as SEQ ID NO. 9 and SEQ ID NO. 10.
Enhancement of transhydrogenase, which is coded by the genes pntA and pntB, as described, for example, in EP 0 733 712 A1,
Enhancement of phosphoenol pyruvate synthase, which is coded by the pps gene, as described, for example, in EP 0 877 090 A1,
Enhancement of phosphoenol pyruvate carboxylase, which is coded by the ppc gene, as described, for example, in EP 0 723 011 A1, and
Enhancement of the regulator RseB, which is coded by the rseB gene, as described, for example, in EP 1382685. The regulator RseB has been described by Missiakas et al. (Molecular Microbiology 24(2), 355-371 (1997)), De Las Penas et al. (Molecular Microbiology 24(2): 373-385 (1997)) and Collinet et al. (Journal of Biological Chemistry 275(43): 33898-33904 (2000)). The associated nucleotide or amino acid sequences are available under the Accession Number AE000343 in public databanks.
Enhancement of the galactose proton symporter (=galactose permease), which is coded by the galP gene, as described, for example, in DE 10314618.0. The galP gene and its function have been described by Macpherson et al. (The Journal of Biological Chemistry 258(7): 4390-4396 (1983)) and Venter et al. (The Biochemical Journal 363(Pt 2): 243-252 (2002)). The associated nucleotide or amino acid sequences are available under the Accession Number AE000377 in public databanks.
Ability to be able to use sucrose as a source of carbon. Genetic determinants for sucrose utilization are described in the prior art, for example in FR-A-2559781, by Debabov (In: Proceedings of the IV International Symposium on Genetics of Industrial Microorganisms 1982. Kodansha Ltd, Tokyo, Japan, p 254-258), Smith and Parsell (Journal of General Microbiology 87,129-140 (1975)) and Livshits et al. (In: Conference on Metabolic Bacterial Plasmids. Tartusk University Press, Tallin, Estonia (1982), p 132-134 and 144-146) and in U.S. Pat. No. 5,705,371. The genetic determinants for sucrose utilization by the strain H155 described by Smith and Parsell were transferred by conjugation into a mutant of Escherichia coli K-12 which is resistant to nalidixic acid and the corresponding transconjugants were deposited on 16th March 2004 at the Deutsche Sammlung fur Mikroorganismen und Zellkulturen [German Collection of Microorganisms and Cell Cultures] (Braunschweig, Germany) as DSM 16293. Genetic determinants for sucrose utilization are also contained in the strain 472T23, which is described in U.S. Pat. No. 5,631,157 and is obtainable from the ATCC under the name ATCC 9801. A further genetic determinant for sucrose utilization has been described by Bockmann et al. (Molecular and General Genetics 235, 22-32 (1992)) and is known under the name csc system.
Enhancement of the YedA gene product, which is coded by the open reading frame yeda, as described, for example, in WO 03/044191.
Growth in the presence of at least 0.1 to 0.5 mM or at least 0.5 to 1 mM borrelidin (borrelidin resistance), as described in U.S. Pat. No. 5,939,307. The borrelidin-resistant strain kat-13 is obtainable under the Accession Number NRRL B-21593 from the NRRL.
Growth in the presence of at least 2 to 2.5 g/l or at least 2.5 to 3 g/l diaminosuccinic acid (diaminosuccinic acid resistance), as described in WO 00/09661. The diaminosuccinic acid-resistant strain DSM 9806 is obtainable under the Accession Number KCCM-10133 from the KCCM.
Growth in the presence of at least 30 to 40 mM or at least 40 to 50 mM α-methylserine (α-methylserine resistance), as described in WO 00/09661. The α-methylserine-resistant strain DSM 9806 is obtainable under the Accession Number KCCM-10133 from the KCCM.
Growth in the presence of not more than 30 mM or not more than 40 mM or not more than 50 mM fluoropyruvic acid (fluoropyruvic acid sensitivity), as described in WO 00/09661. The fluoropyruvic acid-sensitive strain DSM 9806 is obtainable under the Accession Number KCCM-10133 from the KCCM.
Growth in the presence of at least 210 mM or at least 240 mM or at least 270 mM or at least 300 mM L-glutamic acid (glutamic acid resistance), as described in WO 00/09660. The glutamic acid-resistant strain DSM 9807 is obtainable under the Accession Number KCCM-10132 from the KCCM.
An at least partial need for methionine. A strain with an at least partial need for methionine is, for example, the strain H-4257, which is described in U.S. Pat. No. 5,017,483 and is obtainable under the Accession Number FERM BP-984 from the National Institute of Advanced Industrial Science and Technology. The need can be compensated by addition of at least 25, 50 or 100 mg/l L-methionine.
An at least partial need for m-diaminopimelic acid. A strain with an at least partial need for m-diaminopimelic acid is, for example, the strain H-4257, which is described in U.S. Pat. No. 5,017,483 and is obtainable under the Accession Number FERM BP-984 from the National Institute of Advanced Industrial Science and Technology. The need can be compensated by addition of at least 25, 50 or 100 mg/l m-diaminopimelic acid.
Growth in the presence of at least 100 mg/l rifampicin (rifampicin resistance), as described in U.S. Pat. No. 4,996,147. The rifampicin-resistant strain H-4581 is obtainable under the Accession Number FERM BP-1411 from the National Institute of Advanced Industrial Science and Technology.
Growth in the presence of at least 15 g/l L-lysine (lysine resistance), as described in U.S. Pat. No. 4,996,147. The L-lysine-resistant strain H-4581 is obtainable under the Accession Number FERM BP-1411 from the National Institute of Advanced Industrial Science and Technology.
Growth in the presence of at least 15 g/l methionine (methionine resistance), as described in U.S. Pat. No. 4,996,147. The methionine-resistant strain H-4581 is obtainable under the Accession Number FERM BP-1411 from the National Institute of Advanced Industrial Science and Technology.
Growth in the presence of at least 15 g/l L-aspartic acid (aspartic acid resistance), as described in U.S. Pat. No. 4,996,147. The aspartic acid-resistant strain H-4581 is obtainable under the Accession Number FERM BP-1411 from the National Institute of Advanced Industrial Science and Technology.
Enhancement of pyruvate carboxylase, which is coded by the pyc gene. Suitable pyc genes or alleles are, for example, those from Corynebacterium glutamicum (WO 99/18228, WO 00/39305 and WO 02/31158), Rhizobium etli (U.S. Pat. No. 6,455,284) or Bacillus subtilis (EP 1092776). If appropriate, the pyc gene from further microorganisms which contain pyruvate carboxylase endogenously, such as, for example, Methanobacterium thermoautotrophicum or Pseudomonas fluorescens, can also be used.

If sucrose-containing nutrient media are used, the strains are equipped with genetic determinants for sucrose utilization.

The term “enhancement” in this connection describes the increase in the intracellular activity or concentration of one or more enzymes or proteins in a microorganism which are coded by the corresponding DNA, for example by increasing the number of copies of the open reading frame, gene or allele or open reading frames, genes or alleles by at least one (1) copy, using a potent promoter or a gene or allele which codes for a corresponding enzyme or protein with a high activity, and optionally combining these measures.

For the enhancement measures and also for the attenuation measures, the use of endogenous genes, alleles or open reading frames is in general preferred. “Endogenous genes” or “endogenous nucleotide sequences” are understood as meaning the genes or open reading frames or alleles or nucleotide sequences present in the population of a species.

If plasmids are used to increase the number of copies, these are stabilized, if appropriate, by one or more of the genetic loci chosen from the group consisting of the parB locus of the plasmid R1 described by Rasmussen et al. (Molecular and General Genetics 209 (1), 122-128 (1987)), Gerdes et al. (Molecular Microbiology 4 (11), 1807-1818 (1990)) and Thistedt and Gerdes (Journal of Molecular Biology 223 (1), 41-54 (1992)), the flm locus of the F plasmid described by Loh et al. (Gene 66 (2), 259-268 (1988)), the par locus of the plasmid pSC101 described by Miller et al. (Gene 24 (2-3), 309-315 (1983), the cer locus of the plasmid ColE1 described by Leung et al. (DNA 4 (5), 351-355 (1985), the par locus of the plasmid RK2 described by Sobecky et al. (Journal of Bacteriology 178 (7), 2086-2093 (1996)) and Roberts and Helinsky (Journal of Bacteriology 174 (24), 8119-8132 (1992)), the par locus of the plasmid RP4 described by Eberl et al. (Molecular Microbiology 12 (1), 131-141 (1994))and the parA locus of the plasmid R1 described by Gerdes and Molin (Journal of Molecular Biology 190 (3), 269-279 (1986)), Dam and Gerdes (Journal of Molecular Biology 236 (5), 1289-1298 (1994)) and Jensen et al (Proceedings of the National Academy of Sciences USA 95 (15), 8550-8555 (1998).

By enhancement measures, in particular overexpression, the activity or concentration of the corresponding protein or enzyme is in general increased by at least 10%, 25%, 50%, 75%, 100%, 150%, 200%, 300%, 400% or 500%, up to a maximum of 1,000% or 2,000%, based on that of the wild-type protein or the activity or concentration of the protein in the starting microorganism.

To achieve an enhancement, for example, expression of the genes or the catalytic or functional properties of the enzymes or proteins can be increased. The two measures can optionally be combined.

Thus, for example, the number of copies of the corresponding genes can be increased by at least one (1), or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative L-threonine production. The expression is likewise improved by measures to prolong the life of the m-RNA. Furthermore, the enzyme activity is also increased by preventing the degradation of the enzyme protein. The genes or gene constructs can either be present in plasmids with a varying number of copies, or can be integrated and amplified in the chromosome. Alternatively, an overexpression of the genes in question can furthermore be achieved by changing the composition of the media and the culture procedure.

The term “attenuation” in this connection describes the reduction or elimination of the intracellular activity or concentration of one or more enzymes or proteins in a microorganism which are coded by the corresponding DNA, for example by using a weak promoter or an open reading frame or a gene or allele which codes for a corresponding enzyme or protein with a low activity or inactivates the corresponding enzyme or protein or gene and optionally combining these measures.

By attenuation measures, the activity or concentration of the corresponding protein or enzyme is in general reduced to 0 to 75%, 0 to 50%, 0 to 25%, 0 to 10% or 0 to 5% or 0 to 1% or 0 to 0.1% of the activity or concentration of the wild-type protein or of the activity or concentration of the protein in the starting microorganism.

To achieve an attenuation, for example, expression of the genes or open reading frames or the catalytic or functional properties of the enzymes or proteins can be reduced or eliminated. The two measures can optionally be combined.

The reduction in gene expression can take place by suitable culturing, by genetic modification (mutation) of the signal structures of gene expression or also by the antisense-RNA technique. Signal structures of gene expression are, for example, repressor genes, activator genes, operators, promoters, attenuators, ribosome binding sites, the start codon and terminators. The expert can find information in this respect, inter alia, for example, in Jensen and Hammer (Biotechnology and Bioengineering 58: 191-195 (1998)), in Carrier und Keasling (Biotechnology Progress 15: 58-64 (1999)), Franch and Gerdes (Current Opinion in Microbiology 3: 159-164 (2000)) and in known textbooks of genetics and molecular biology, such as, for example, the textbook by Knippers (“Molekulare Genetik [Molecular Genetics]”, 6th edition, Georg Thieme Verlag, Stuttgart, Germany, 1995) or that by Winnacker (“Gene und Klone [Genes and Clones]”, VCH Verlagsgesellschaft, Weinheim, Germany, 1990).

Mutations which lead to a change or reduction in the catalytic properties of enzyme proteins are known from the prior art. Examples which may be mentioned are the works of Qiu and Goodman (Journal of Biological Chemistry 272: 8611-8617 (1997)), Yano et al. (Proceedings of the National Academy of Sciences of the United States of America 95: 5511-5515 (1998)), Wente and Schachmann (Journal of Biological Chemistry 266: 20833-20839 (1991)). Summarizing descriptions can be found in known textbooks of genetics and molecular biology, such as e.g. that by Hagemann (“Allgemeine Genetik [General Genetics]”, Gustav Fischer Verlag, Stuttgart, 1986).

Possible mutations are transitions, transversions, insertions and deletions of at least one (1) base pair or nucleotide. Depending on the effect of the amino acid exchange caused by the mutation on the enzyme activity, “missense mutations” or “nonsense mutations” are referred to. Missense mutation leads to an exchange of a given amino acid in a protein for another, this being, in particular, a non-conservative amino acid exchange. The functional capacity or activity of the protein is impaired by this means and reduced to a value of 0 to 75%, 0 to 50%, 0 to 25%, 0 to 10%, 0 to 5%, 0 to 1% or 0 to 0.1%. Nonsense mutation leads to a stop codon in the coding region of the gene and therefore to a premature interruption in the translation. Insertions or deletions of at least one base pair in a gene lead to frame shift mutations, which lead to incorrect amino acids being incorporated or translation being interrupted prematurely. If a stop codon is formed in the coding region as a consequence of the mutation, this also leads to a premature termination of the translation. Deletions of at least one (1) or more codons typically also lead to a complete loss of the enzyme activity or function.

Strains which are suitable for the process according to the invention are, inter alia, the strain BKIIM B-3996 described in U.S. Pat. No. 5,175,107, the strain KCCM-10132 described in WO 00/09660, and isoleucine-needing mutants of the strain kat-13 described in WO 98/04715. If appropriate, strains can be adapted to the process according to the invention with the measures mentioned, in particular by incorporation of a stop codon into the rpoS gene, for example an amber codon at the site corresponding to position 33 of the amino acid sequence of the RpoS protein, and simultaneous incorporation of a corresponding t-RNA suppressor, for example supE.

Strains which are suitable for the process according to the invention can also be identified by determining the nucleotide sequence of the rpoS gene in a strain of Escherichia coli which secretes L-threonine. For this purpose, the rpoS gene is cloned, or amplified with the aid of the polymerase chain reaction (PCR) and the nucleotide sequence is determined. If the rpoS gene contains a stop codon, it is checked in a second step whether it also contains a corresponding t-RNA suppressor. If appropriate, the strain identified in this manner is provided with the properties described above, such as, for example, overexpression of the thrA allele, attenuation of the threonine breakdown which takes place under aerobic culture conditions, introduction into the ilvA gene of a mutation which effects an at least partial need for isoleucine or growth in the presence of at least 5 g/l threonine, or with one or more of the properties furthermore listed.

The properties or features mentioned can be transferred into desired strains by transformation, transduction or conjugation.

In the method of transformation, isolated genetic material, typically DNA, is inserted into a recipient strain. In the case of bacteria of the Enterobacteriaceae family, such as e.g. Escherichia coli, the DNA for this purpose is incorporated into plasmid or phage DNA in vitro and this is then transferred into the recipient strain. The corresponding methods and working instructions are adequately known in the prior art and are described in detail, for example, in the handbook by J. Sambrook (Molecular Cloning, A Laboratory Manual, 2nd ed., Cold Spring Harbor Laboratory Press, Cold Spring Harbor, N.Y., 1989).

Defined mutation can be transferred into suitable strains with the aid of the method of gene or allele exchange using conditionally replicating plasmids. In the case of a defined mutation, at least the position in the chromosome, preferably the exact position of the change in the nucleobase(s) and the nature of the change (substitution, i.e. transition or transversion, insertion or deletion), is known. If appropriate, the corresponding DNA is initially sequenced with the usual methods. A usual method for achieving a gene or allele exchange is that described by Hamilton et al. (Journal of Bacteriology 171: 4617-4622 (1989)), in which the pSC101 derivative pMAK705, which replicates sensitively to heat, is used. Alleles can be transferred from the plasmid into the chromosome with this method. Chromosomal alleles can be transferred to the plasmid in the same manner. Other methods described in the prior art, such as, for example, that of Martinez-Morales et al. (Journal of Bacteriology 181: 7143-7148 (1999)), that of Boyd et al. (Journal of Bacteriology 182: 842-847 (2000)) or the method described in WO 01/77345 can likewise be used.

This method can be employed, inter alia to insert rpoS alleles which contain, for example, stop codons, suppressor genes, such as, for example, supE, attenuated tdh alleles which contain, for example, deletions, attenuated ilvA alleles, thrA alleles which code for “feed back” resistant aspartate kinase I—homoserine dehydrogenase I variants, the rhtA23 mutation, attenuated pck alleles, attenuated alleles of the ytfp ORF, attenuated yjfA ORFs, attenuated poxB alleles or attenuated yjgF ORFs into desired strains.

In the method of transduction, a genetic feature is transferred from a donor strain into a recipient strain using a bacteriophage. This method belongs to the prior art and is described in textbooks such as, for example, that of E. A. Birge (Bacterial and Bacteriophage Genetics, 4th ed., Springer Verlag, New York, USA, 2000).

In the case of Escherichia coli, the bacteriophage P1 is typically used for generalized transduction (Lennox, Virology 1, 190-206 (1955)). A summary of the methods of generalized transduction is given in the article “Generalized Transduction” by M. Masters contained in the textbook by F. C. Neidhard (Escherichia coli and Salmonella Cellular and Molecular Biology, 2nd ed., ASM Press, Washington, D.C., USA, 1996). Practical instructions, for example, are contained in the handbook by J. H. Miller (A Short Course In Bacterial Genetics. A Laboratory Manual and Handbook for Escherichia coli and Related Bacteria, Cold Spring Harbor Laboratory Press, New York, USA, 1992) or the handbook by P. Gerhardt “Manual of Methods for General Bacteriology” (American Society for Microbiology, Washington, D.C., USA, 1981).

Resistance-imparting or other dominant genetic properties, such as, for example, antibiotics resistance (for example kanamycin resistance, chloramphenicol resistance, rifampicin resistance or borrelidin resistance), resistance to antimetabolites (for example α-amino-β-hydroxyvaleric acid resistance, α-methyl-serine resistance or diaminosuccinic acid resistance), resistance to metabolites (for example threonine resistance, homoserine resistance, glutamic acid resistance, methionine resistance, lysine resistance or aspartic acid resistance) or also the ability to utilize sucrose, can be transferred into suitable recipient strains with the aid of transduction.

The method of transduction is also suitable for inserting so-called non-selectable genetic properties, such as, for example, auxotrophies or needs for amino acids (for example the need for isoleucine, the need for methionine or the need for m-diaminopimelic acid), needs for vitamins or sensitivity to antimetabolites (for example fluoropyruvic acid sensitivity) into recipient strains. E. coli strains which contain the transposon Tn10 or Tn10kan in an interval of approximately one minute on the chromosome are used for this purpose. These strains are known under the term “Singer Collection” or “Singer/Gross Collection” (Singer et al., Microbiological Reviews 53, 1-24, 1989). These strains are generally available from the E. coli Genetic Stock Center of Yale University (New Haven, Conn., USA). Further information is to be found in the article by M. K. B. Berlyn et al. “Linkage Map of Escherichia coli K-12, Edition 9” contained in the textbook by F. C. Neidhard (Escherichia coli and Salmonella Cellular and Molecular Biology, 2nd ed., ASM Press, Washington, D.C., USA, 1996). In a similar manner, genetic properties which are not directly selectable (for example fluoropyruvic acid sensitivity, suppressor mutations) and also those whose mutation site is not known, can be transferred into various strains. Instructions in this context are to be found inter alia in the textbook by J. Scaife et al. (Genetics of Bacteria, Academic Press, London, UK, 1985), in the above-mentioned article by M. Masters and in the above-mentioned handbook by J. H. Miller. The tetracycline resistance gene inserted with the transposon Tn10 can optionally be removed again with the method described by Bochner et al. (Journal of Bacteriology 143, 926-933 (1980)).

In the method of conjugation, genetic material is transferred from a donor into a recipient by cell-cell contact. Conjugative transfer of the F factor (F: fertility), conjugative gene transfer using Hfr strains (Hfr: high frequency of recombination) and strains which carry an F′ factor (F′: F prime) belong to the conventional methods of genetics. Summarizing descriptions are to be found, inter alia, in the standard work by F. C. Neidhard (Escherichia coli and Salmonella Cellular and Molecular Biology, 2nd ed., ASM Press, Washington, D.C., USA, 1996). Practical instructions, for example, are contained in the handbook by J. H. Miller (A Short Course In Bacterial Genetics. A Laboratory Manual and Handbook for Escherichia coli and Related Bacteria, Cold Spring Harbor Laboratory Press, New York, USA, 1992) or the handbook by P. Gerhardt “Manual of Methods for General Bacteriology” (American Society for Microbiology, Washington, D.C., USA, 1981). F, F′ and Hfr strains are generally available from the E. coli Genetic Stock Center of Yale University (New Haven, Conn., USA). The method of conjugation has been employed, for example, to transfer the mutation thrC1010 described by Theze and Saint-Girons (Journal of Bacteriology 118, 990-998 (1974)) into the strain MG442 (Debabov, Advances in Biochemical Engineering/Biotechnology 79, 113-136 (2003). Conjugative plasmids which carry the ability to utilize sucrose are described in the prior art, for example by Schmid et al. (Journal of Bacteriology 151, 68-76 (1982)) or Smith and Parsell (Journal of General Microbiology 87, 129-140 (1975)) and Livshits et al. (In: Conference on Metabolic Bacterial Plasmids. Tartusk University Press, Tallin, Estonia (1982), p 132-134 und 144-146). Thus, Debabov reports (In: Proceedings of the IVth International Symposium on Genetics of Industrial Microorganisms 1982. Kodansha Ltd, Tokyo, Japan, p 254-258) on the construction of threonine-producing strains into which the ability to utilize sucrose has been incorporated with the aid of conjugation.

1-38. (canceled)

39. A process for the preparation of L-threonine using bacteria of the Enterobacteriaceae family which produce L-threonine, comprising:
a) inoculating and culturing a bacterium of the Enterobacteriaceae family in at least a first nutrient medium;
b) feeding at least one additional nutrient medium into the culture of step a) in one or more feed streams, wherein:
i) said additional nutrient medium comprises at least one source of carbon, at least one source of nitrogen and at least one source of phosphorus;
ii) said culture is maintained under conditions which allow the formation of L-threonine;
iii) at the same that said additional nutrient medium is added, removing culture broth from the culture in one or several removal streams wherein the total flow rate of said one or more removal streams is substantially the same as the total flow rate of said one or more feed streams, and wherein
c) the concentration of the source of carbon during the continuous culturing in step b) is adjusted to not more than 30 g/l.

40. The process of claim 39, wherein the culturing in step (a) is carried out by a batch process.

41. The process of claim 39, wherein the culturing of step (a) is carried out by a fed batch process, in which at least one additional nutrient medium is employed.

42. The process of claim 39, further comprising purifying L-threonine from said culture broth.

43. The process of claim 39, further comprising treating the culture broth removed in said one or several removal streams by:
a) removing at least 90% of the biomass present; and
b) removing at least 90% of the water remaining in said removal stream after the removal of said biomass.

44. The process of claim 39, wherein said source of carbon is one or more compounds selected from the group consisting of: sucrose; molasses from sugar beet or cane sugar; fructose; glucose; starch hydrolysate; cellulose hydrolysate; arabinose; maltose; xylose; acetic acid; ethanol; and methanol.

45. The process of claim 39, wherein said source of nitrogen is one or more organic nitrogen-containing substances or substance mixtures selected from the group consisting of: peptones; yeast extracts; meat extracts; malt extracts; corn steep liquor; soya bean flour; and urea; and/or one or more of the inorganic compounds selected from the group consisting of: ammonia; ammonium-containing salts; and salts of nitric acid.

46. The process of claim 39, wherein said source of nitrogen is selected from the group consisting of: ammonium sulfate; ammonium chloride; ammonium phosphate; ammonium carbonate; ammonium nitrate; potassium nitrate; and potassium sodium nitrate.

47. The process of claim 39, wherein said source of phosphorus is either:
a) phosphoric acid or polymers thereof; or
b) phytic acid or alkali metal or alkaline earth metal salts thereof.

48. The process of claim 39, wherein said source of phosphorus is selected from the group consisting of: potassium dihydrogen phosphate; dipotassium hydrogen phosphate; and the corresponding sodium-containing salts thereof.

49. The process of claim 39, wherein said bacteria of the Enterobacteriaceae family contain at least one thrA gene or allele which codes for a threonine-insensitive aspartate kinase I—homoserine dehydrogenase I.

50. The process of claim 39, wherein said bacteria of the Enterobacteriaceae family contains a stop codon selected from the group consisting of: opal; ochre; and amber; in the rpoS gene and a t-RNA suppressor selected from the group consisting of: opal suppressor; ochre suppressor; and amber suppressor.

51. The process of claim 39, wherein said nutriant feed medium has a phosphorus to carbon ratio (P/C ratio) selected from: not more than 4; not more than 3; not more than 2; not more than 1.5; not more than 1; not more than 0.7; not more than 0.5; not more than 0.48; not more than 0.46; not more than 0.44; not more than 0.42; not more than 0.40; not more than 0.38; not more than 0.36; not more than 0.34; not more than 0.32; and not more than 0.30.

52. The process of claim 39, wherein the culture broth removed is provided with oxygen or an oxygen-containing gas until the concentration of the source of carbon falls below a value selected from: 2 g/l; 1 g/l; and 0.5 g/l.

53. The process of claim 50, wherein the L-threonine formed is purified.

54. The process of claim 50, further comprising treating the culture broth removed in said one or several removal streams by:
a) removing at least 90% of the biomass present; and
b) removing at least 90% of the water remaining in said removal stream after the removal of said biomass.

55. The process of claim 39, wherein the concentration of the source of carbon during the culture is adjusted to a value selected from: not more than 20; not more than 10; not more than 5 g/l and not more than 2 g/l.

56. The process of claim 39, wherein the yield of L-threonine formed, based on the source of carbon employed, is selected from a value of: at least 31%; at least 37%; and at least 42%.

57. The process of claim 39, wherein L-threonine is formed with a space/time yield having a value selected from: at least 1.5 to 2.5 g/l per h; 2.5 to more than 3.5 g/l per h; at least 3.5 to 5.0 g/l per h; at least 5.0 to more than 8.0 g/l per h.

58. Sucrose-utilizing transconjugants of Escherichia coli K-12 deposited as DSM 16293 at the Deutsche Sammlung für Mikroorganismen und Zellkulturen [German Collection of Microorganisms and Cell Cultures] (Braunschweig, Germany).

59. The process of claim 39, wherein said bacteria of the Enterobacteriaceae family is a strain which has one or more of the following features:
a) a threonine-insensitive aspartate kinase I—homoserine dehydrogenase I, which is optionally present in overexpressed form;
b) a stop codon selected from the group consisting of: opal, ochre and amber in the rpoS gene; and a t-RNA suppressor selected from the group consisting of:
opal suppressor, ochre suppressor and amber suppressor;

60. The process of claim 59, wherein said strain further comprises one or more of the following features:
a) an incapablitiy, under aerobic culture conditions, of breaking down threonine,
b) at least a partial need for isoleucine, and
c) a capacity to grow in the presence of at least 5 g/l threonine.

61. The process of claim 59, wherein said strain further comprises one or more of the following features:
a) attenuation of phosphoenol pyruvate carboxykinase, which is coded for by the pckA gene;
b) attenuation of phosphoglucose isomerase, which is coded for by the pgi gene;
c) attenuation of the YtfP gene product, which is coded for by the open reading frame ytfP;
d) attenuation of the YjfA gene product, which is coded for by the open reading frame yjfA;
e) attenuation of pyruvate oxidase, which is coded for by the poxB gene;
f) attenuation of the YjgF gene product, which is coded for by the open reading frame yjgF;
g) enhancement of transhydrogenase, which is coded for by the genes pntA and pntB;
h) enhancement of phosphoenol pyruvate synthase, which is coded for by the pps gene;
i) enhancement of phosphoenol pyruvate carboxylase, which is coded for by the ppc gene;
j) enhancement of the regulator RseB, which is coded for by the rseB gene;
k) enhancement of the galactose proton symporter, which is coded for by the galP gene;
l) an ability to use sucrose as a source of carbon;
m) enhancement of the YedA gene product, which is coded for by the open reading frame yedA;
n) growth in the presence of at least 0.1 to 0.5 mM or at least 0.5 to 1 mM borrelidin (borrelidin resistance);
o) growth in the presence of at least 2 to 2.5 g/l or at least 2.5 to 3 g/l diaminosuccinic acid (diaminosuccinic acid resistance);
p) growth in the presence of at least 30 to 40 mM or at least 40 to 50 mM α-methylserine (α-methylserine resistance);
q) growth in the presence of not more than 30 mM or not more than 40 mM or not more than 50 mM fluoropyruvic acid (fluoropyruvic acid sensitivity);
r) growth in the presence of at least 210 mM or at least 240 mM or at least 270 mM or at least 300 mM L-glutamic acid (glutamic acid resistance);
s) at least a partial need for methionine;
t) at least a partial need for m-diaminopimelic acid;
u) growth in the presence of at least 100 mg/l rifampicin (rifampicin resistance);
v) growth in the presence of at 15 g/l L-lysine (lysine resistance);
w) growth in the presence of at least 15 g/l methionine (methionine resistance);
x) growth in the presence of at least 15 g/l L-aspartic acid (aspartic acid resistance); or
enhancement of pyruvate carboxylase, which is coded for by the pyc gene.
